(19) United States
(12) Patent Application Publication
(10) Pub. No.: US 2013/0255670 A1
(43) Pub. Date: Oct. 3, 2013

(54) System and Method for Performing a Surgical Procedure

(71) Applicant: LEXION MEDICAL, LLC, St. Paul, MN (US)

(72) Inventors: Douglas E. Ott, Macon, GA (US); Nathaniel V. Tran, Apple Valley, MN (US); Steven B. Williams, White Bear Lake, MN (US); Brandon Lee Michal, White Bear Lake, MN (US)

(73) Assignee: Lexion Medical, LLC, St. Paul, MN (US)

(21) Appl. No.: 13/854,682

(22) Filed: Apr. 1, 2013

Related U.S. Application Data
(60) Provisional application No. 61/619,122, filed on Apr. 2, 2012.

(51) Int. Cl. A61M 11/02

(52) U.S. Cl. CPC A61M 11/02
USPC 128/200.14

(57) ABSTRACT

In accordance with one embodiment of the present disclosure, a method for performing a surgical procedure may include atomizing at least a portion of a humidification liquid. The method may also include receiving a gas from a source. The method may further include generating a vortex flow of the received gas. The method may further include combining the vortex flow of the received gas with at least a portion of the atomized humidification liquid in order to humidify the received gas. The method may further include providing the humidified gas adjacent to or into a patient.

### RELATED APPLICATION

This application claims the benefit under 35 U.S.C.§119(e) of the priority of U.S. Provisional Application No. 61/619,122 filed Apr. 2, 2012, entitled “System And Method For Performing A Surgical Procedure.”

### TECHNICAL FIELD

This disclosure relates in general to surgery and more particularly to a system and method for performing a surgical procedure.

### BACKGROUND

Traditional systems and methods for performing a surgical procedure sometimes involve providing a gas adjacent to or into the patient. One negative to the use of such gases in a surgery is that gases are dry and have the potential to lead to various states of desiccation and tissue damage. Various systems and methods have been developed to humidify and/or warm the gas prior to providing it adjacent to or into the patient. These prior art systems, however, have disadvantages which may be addressed by the invention.

### SUMMARY OF THE DISCLOSURE

In accordance with one embodiment of the present disclosure, a method for performing a surgical procedure may include atomizing at least a portion of a humidification liquid. The method may also include receiving a gas from a source. The method may further include generating a vortex flow of the received gas. The method may further include combining the vortex flow of the received gas with at least a portion of the atomized humidification liquid in order to humidify the received gas. The method may further include providing the humidified gas adjacent to or into a patient.

Numerous technical advantages are provided according to various embodiments of the present disclosure. Particular embodiments of the disclosure may exhibit none, some, or all of the following advantages depending on the implementation.

In particular embodiments, atomizing (or nebulizing) the humidification liquid may greatly increase the likelihood that the compound that is atomized will remain a part of the humidified gas. In particular embodiments, by generating a vortex flow in order to humidify the gas, the gas may be adequately and efficiently conditioned. The vortex flow may increase the humidification of the gas because it may increase the amount of atomized humidification liquid pulled to the top of the humidification chamber, where it may more adequately and efficiently condition the gas. Additionally, in particular embodiments, by warming and/or vaporizing the atomized humidification liquid after the humidified gas enters a delivery conduit, the likelihood of a medicant dropping out of the gas may be reduced. Furthermore, in particular embodiments, by vaporizing (either completely or partially) an atomized humidification liquid, obstruction of the vision of the surgeon performing a medical procedure may be prevented (or the amount of such obstruction may be decreased). Thus, the invention may provide both a way to effectively humidify gas and to use that gas to deliver a drug effectively in vapor form while reducing the obstruction of the user's vision.

Other technical advantages of the present disclosure will be readily apparent to one skilled in the art from the following figures, descriptions, and claims. Moreover, while specific advantages have been enumerated above, various embodiments may include all, some, or none of the enumerated advantages.

### BRIEF DESCRIPTION OF THE DRAWINGS

For a more complete understanding of the present disclosure and its advantages, reference is now made to the following description, taken in conjunction with the accompanying drawings, in which:

FIG. 1 is a schematic view of one embodiment of an system that may be used to perform a surgical procedure;

FIG. 2 is a schematic view of one embodiment of a device of the system of FIG. 1 that may be used to perform a surgical procedure; and

FIG. 3 illustrates one embodiment of a method for performing a surgical procedure.

### DETAILED DESCRIPTION OF THE DRAWINGS

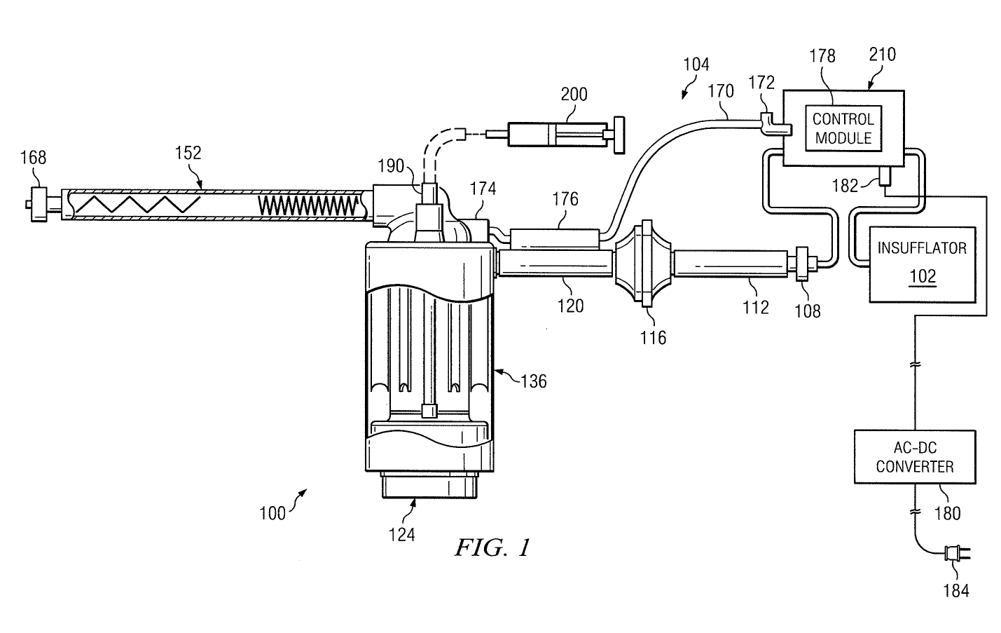

Embodiments of the present disclosure and their advantages are best understood by referring to FIGS. 1 through 3 of the drawings, like numerals being used for like and corresponding parts of the various drawings.

FIG. 1 illustrates one embodiment of a system 100 for performing a surgical procedure. According to the illustrated embodiment, system 100 includes a device 104 for warming and/or humidifying gas. Optionally, the gas may be humidified with a medicant, rather than simply with water vapor or atomized water of some type. In order to humidify the gas, device 104 may atomize (or nebulize) the humidification liquid. In particular embodiments, atomizing (or nebulizing) the humidification liquid may greatly increase the likelihood that the compound that is atomized will remain a part of the humidified gas. Furthermore, device 104 may humidify the gas by generating a vortex flow of the gas. In particular embodiments, such a vortex flow of the gas may allow the gas to be adequately and efficiently conditioned. Higher and more consistent levels of humidity may be obtained because the vortex flow may increase the amount of atomized humidification liquid pulled to the top of the humidification chamber, where it may more adequately and efficiently condition the gas. Additionally, device 104 may also warm and/or vaporize the atomized humidification liquid after the humidified gas enters the delivery conduit 152 of the device 104. In particular embodiments, by warming and/or vaporizing the atomized humidification liquid after the humidified gas enters a delivery conduit, the likelihood of a medicant dropping out of the gas may be reduced. Furthermore, in particular embodiments, by vaporizing (either completely or partially) an atomized humidification liquid, obstruction of the vision of the surgeon performing a medical procedure may be prevented (or the amount of such obstruction may be decreased). Thus, the invention may provide both a way to effectively humidify gas and to use that gas to deliver a drug effectively in vapor form while reducing the obstruction of the user's vision.

A surgical procedure refers to any surgical procedure that may utilize warmed and/or humidified gas. For example, the procedure may be an endoscopic surgical procedure, such as laparoscopy, colonoscopy, gastroscopy, bronchoscopy, and/or thoracoscopy. As another example, the procedure may be an open surgical site procedure, such as a laparotomy, thoracotomy, craniotomy, traumatic wound procedure, cardiac surgery procedure, chest surgery procedure, brain surgery procedure, ENT (ear, nose, and/or throat) surgery procedure, bladder or Cesarean Section procedure, or any other open site surgery. As a further example, the procedure may be any procedure that may utilize warmed and/or humidified gas. In particular embodiments, a surgical procedure may further include any procedure that utilizes warmed and/or humidified oxygen or any anesthetic gases or combination of gases for breathing, for example, or to administer anesthesia or breathing therapy. The surgical procedure may be performed on any patient. In one embodiment, the patient may be a human being. In another embodiment, the patient may be an animal, such as a dog, cat, horse, pig, or any other animal.

The gas received by device 104 of system 100 may include any suitable gas. For example, the gas may include carbon dioxide, oxygen, nitrous oxide, argon, helium, nitrogen, room air, or inert gases. In a further embodiment, the gas may include a combination of gases. For example, the gas may include a combination of carbon dioxide and nitrous oxide. Preferable gases for endoscopy are carbon dioxide and nitrous oxide. A combination of the above gases can be used, i.e., 100% of a single gas need not be used. The gas may be received from any suitable source. For example, the gas may be received from an insufflator, such as insufflator 102. As another example, the gas may be received from any other source that may provide a gas, such as, for example, a gas cartridge, a gas pump, a tank with a flow regulator, a centralized gas supply system in a hospital, or any other gas source.

The humidification liquid used to humidify the gas may refer to any liquid for a surgical procedure. For example, the liquid may include water, such as sterile water. As another example, the liquid may include saline. In a further embodiment the liquid may include an anesthetic, antibiotic, or both. For example, the liquid may include lidocaine. In a further embodiment, the liquid may include an anticoagulant in order to prevent blood clots or clotting. For example, the liquid may include heparin or Angiomax. In a further embodiment, the liquid may include any other medicant or pharmacologic agent. For example, the liquid may include an antihypertensive, an anti-adhesive agent, and/or a chemotherapeutic agent. The liquid may also include a combination of water (or saline) and other substances, such as anesthetics, antibiotics, or anticoagulants. In particular embodiments, the liquid may include any physiologic compatible therapeutic solution, such as 1XTted Ringers, Hartmans, or bicarbonate solution.

According to the illustrated embodiments, device 104 is adapted to receive gas from a gas source (high or low pressure, high or low flow rate), such as insufflation gas from an insufflator 102 for delivery adjacent to or into a patient. The device 104 comprises a filter 116, an atomizer 124, a humidification chamber 136, and a control module 178. Filter 116 is optional, but preferable. A tubing set (or conduit set) is provided to connect the various components of device 104 together. Specifically, a first tube segment 112 connects the outlet of a control housing 210 (which receives gas from insufflator 102 and, based on control module 178, provides the gas to the outlet) to the inlet tubing of the filter 116 via a male Luer lock 108 or any appropriate adaptor compatible with the outlet port. In a further embodiment, the insufflator 102 may include control module 178 (as opposed to control housing 210) and may be able to perform all of the functions discussed below with regard to control module 178. In such an embodiment, tube segment 112 may be attached directly to the outlet of insufflator 102 by the Luer lock 108 or any appropriate adaptor compatible with the outlet port. A second tube segment 120 connects the outlet of the filter 116 to the inlet of the humidification chamber 136. A third tubing set (or delivery conduit 152) connects the outlet of the humidification chamber 136 by a male Luer lock 168 (or other appropriate fitting adaptor) to a gas delivery device (not shown), such as a trocar, verres needle, endoscope, a diffusing gas delivery device, a mouthpiece, a wound bandage delivery system (which includes any system that allows humidified and/or warmed gas that may or may not include a medicant to come in contact with a wound or open sore), or a tube that enters or is adjacent to a body cavity or space that delivers the filtered, warmed, and/or humidified gas adjacent to or into the body of a patient. The tubing of tube segment 112, tube segment 120, and delivery conduit 152 may be flexible and sufficiently long to permit the insufflator 102 and control module 178 to be placed at a convenient distance from a patient, while the humidification chamber 136 may be placed within 12 inches of the patient. In other embodiments, humidification chamber 136 may be placed a greater distance from the patient, such as, for example, 12 feet from the patient. Although tubing is a preferred structure for gas delivery, any fluid conduit could be used between the insufflator and humidification chamber 136.

The filter 116 is optional, but may be a particulate filter (for example a BF201 filter from AG Industries, with a HA-8141 filter media from Hollingsworth & Vose) having a pore size preferably small enough to exclude all solid particles and bacterial or fungal agents that may have been generated in or on a gas supply cylinder, such as a carbon dioxide cartridge, or the insufflator 102 (e.g., 0.5 micron or less, for example, about 0.2 micron). As another example, the filter 116 may be a DDF5500MO2C-LM particulate filter from Porous Media. As a further example, the filter 116 may be a HWB-FLTR-CO2-1 particulate filter from AG Industries. In one embodiment, the filter 116 is a hydrophobic filter. In another embodiment, the filter 116 is a hydrophilic filter. In a further embodiment, decreasing the pore size of filter 116 below 0.2 micron may cause a concomitant increase in pressure drop of gas, and thus flow rate may be reduced significantly. In particular embodiments, filter 116 may be a 0.2 micron filtration element sandwiched in-between injection molded plastic housings-halves, which are ultrasonically welded together. In further embodiments, filter 116 may take on different configurations, depending on the desired flow characteristics associated with different types of gases and/or surgical procedures. In one embodiment, the filter 116 may be disposed in the device 104 in any suitable location. For example, the filter 116 may be disposed in the device 104 in a location where the gas passes through the filter 116 before entering the humidification chamber 136. As such, the gas may be filtered prior to being humidified.

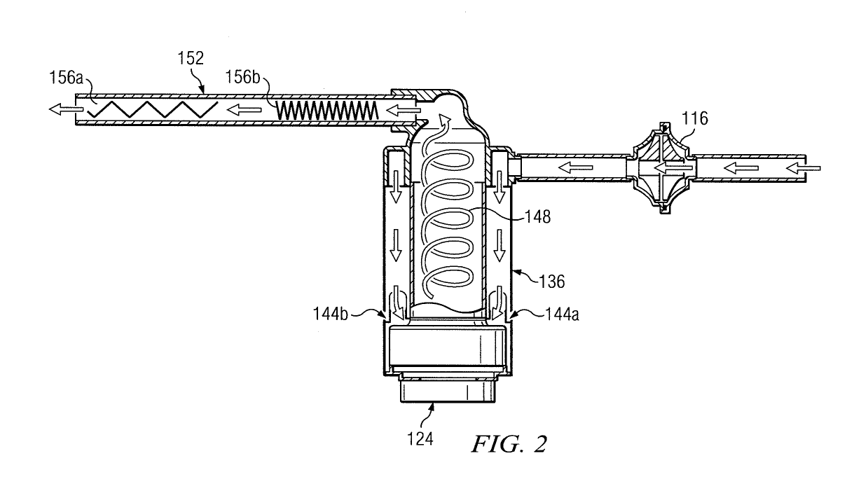

Atomizer 124 may be any component or components operable to atomize (or nebulize) at least a portion of a humidification liquid in humidification chamber 136. Atomizer 124 is described in further detail in FIG. 2.

Humidification chamber 136 may be any chamber operable to receive a gas from a source and humidify the received gas. Humidification chamber 136 is described in further detail in FIG. 2.

Delivery conduit 152 may be any conduit operable to receive the humidified gas from the humidification chamber 136 and provide the humidified gas adjacent to or into a patient. In particular embodiments, delivery conduit 152 may include a heating element. Delivery conduit 152 is described in further detail in FIG. 2.

In one embodiment, the control module 178 is contained within a control housing 210 and is connected to the device 104 by several wire pairs contained within an insulated electrical cable 170. In particular, the cable 170 has a connector 172 at one end that electrically connects into a receptacle of the housing 210 for the control module 178, and at the other end it is electrically connected to the device 104 by a sealed electrical feedthrough 174. In one embodiment, the cable 170 is attached to the tube segment 120 by a plastic tape or clip 176. In another embodiment, the cable 170 is attached to the tube segment 120 by heat seal, extrusion, co-extrusion, ultrasonic welding, laser transmission welding, glue, another solvent bond, or is passed through the interior of tube segment 120.

The control module 178 and associated components in the device 104 may be powered by an AC-DC converter 180. In one embodiment, the AC-DC converter 180 has an output that is connected by a plug connector 182 into a receptacle of the housing 210 to the control module 178, and has a standard AC wall outlet plug 184 that can be plugged into standard AC power outlets. For example, the AC-DC converter 180 is plugged into an AC power strip that is provided on other equipment in an operating room. In another embodiment, electrical power for the apparatus is provided by a battery or photovoltaic source. In further embodiments, circuitry may be provided in the control module 178 that operates on AC signals, as opposed to DC signals, in which case the control module 178 could be powered directly by an AC outlet.

In one embodiment, the device 104 has a charging port 190 that is capable of receiving a supply of liquid therethrough to charge the humidification chamber 136 with humidification liquid. For example, a syringe 200 containing a predetermined volume of humidification liquid is introduced into the charging port 190 to inject liquid into the humidification chamber 136 for an initial charge or recharge of humidification liquid. As another example, humidification liquid may be provided into the charging port 190 and/or the humidification chamber 136 by any other apparatus, such as an Intravenous (IV) bag (or Saline bag) controlled by a pressurized pump system or pressure cuff, a metering system (such as a metered syringe), a peristaltic pump, or any other suitable apparatus. The device 104 may be sold with the humidification chamber 136 pre-charged with a supply of humidification liquid such that an initial charge is not required for operation.

The charging port 190 comprises a cylindrical body containing a resealable member (not shown). The resealable member permits a device (such as syringe 200) to be inserted therethrough, but seals around the exterior of the device. This allows a volume of humidification liquid (sterile water, saline, etc.) to be delivered into the cylindrical body of the charging port 190 without releasing the liquid already contained therein. The resealable member is, for example, a Luer lock check valve, such as P/N B900-SSM41 manufactured by NP Medical or P/N SCV23050 manufactured by Value Plastics. Alternatively, the charging port may be embodied by a one-way valve, a sealable port, a screw cap, a cap with a slit to permit the introduction of a syringe or other device, such as a SAFELINE injection site, part number NF9100, manufactured by B. Braun Medical Inc., or any other covering material or member capable of permitting the introduction of a device and preventing the backflow of contained liquid or gas. In one embodiment, the chamber of charging port 190 may contain approximately 3 to 8 cubic centimeters (cc) (but possibly as much as 50 cc) of liquid. The control module 178, however, may issue a warning when the humidity of the gas being treated by the device 104 drops below a predetermined relative humidity, as explained hereinafter.

Although device 104 of FIG. 1 has been described as including only a single charging port, device 104 may include any number of charging ports. For example, device 104 may include one or more charging ports for supplying humidification chamber 136 with humidification liquid, and may further include one or more charging ports for providing a pharmacologic agent or a medicant (such as an anesthetic or antibiotic) to humidification chamber 136. In such an example, the amount of humidification liquid and medicant provided into humidification chamber 136 may each be controlled independently. For example, while the amount of humidification liquid provided into humidification chamber 136 may remain steady throughout a procedure, the amount or type of medicant provided into humidification chamber 136 may be varied depending on the stage of the procedure or a selection by a user. In particular embodiments, this may allow medicant profiles to be utilized with device 104. For example, during particular points in a procedure, a specific medicant profile (such as, for example, the delivery of 5 mg of a medicant for the next five minutes or for the next 5 liters of gas) may be selected at the control module 178, thereby causing control module 178 to provide the medicant to humidification chamber 136 in accordance with such a medicant profile. In particular embodiments, instead of including one or more separate charging ports for humidification liquid and medicant, a single charging port may be utilized for both the humidification liquid and the medicant. For example, a combining device (such a Y-bracket or any other static mixing feature) may be attached to the charging port, thus enabling both humidification liquid and a medicant to be provided to humidification chamber 136 through the same charging port. In such an example, the combining device may dilute the medicant in the humidification liquid prior to the humidification liquid being provided into humidification chamber 136.

Although device 104 of FIG. 1 has been described above as including a charging port 190 for charging the humidification chamber 136 with humidification liquid, any other charging apparatus or device may be utilized. As an example, device 104 may include a preliminary fill chamber that feeds humidification liquid into humidification chamber 136 whenever an amount of humidification liquid in the humidification chamber 136 drops below a predetermined amount. In particular, as is discussed in further detail below with regard to FIG. 2, device 104 may include a float system that rises and/or falls with the level of the humidification liquid. When the float system falls, the float system may unblock a port that connects the humidification chamber 136 to the preliminary fill chamber, causing the humidification liquid in the preliminary fill chamber to recharge the humidification chamber 136.

Modifications, additions, or omissions may be made to the system 100 without departing from the scope of the invention. The components of the system 100 may be integrated or separated. Moreover, the operations of the system 100 may be performed by more, fewer, or other components. For example, the operations of the humidification chamber 136 may be performed by more than one component.

Turning to FIG. 2, one embodiment of the device 104 will be described in greater detail. According to the illustrated embodiment, device 104 includes atomizer 124, humidification chamber 136, and delivery conduit 152.

As is discussed above with regard to FIG. 1, atomizer 124 may atomize (or nebulize) at least a portion of the humidification liquid in the humidification chamber 136. Atomization (or nebulization) refers to reducing a quantity of humidification liquid into tiny particles, such as a mist, fog, or fine spray.

Atomizer 124 may be any component or components that may atomize (or nebulize) the humidification liquid in humidification chamber 136. For example, atomizer 124 may be one or more piezoelectric elements, such as one or more piezoelectric ceramic discs. As another example, atomizer 124 may be one or more ceramic discs, pneumatic pumps, one or more elements that utilize mechanical agitation in order to atomize the humidification liquid, one or more devices that utilize mechanical shearing to atomize the humidification liquid, one or more elements that utilize any other method for atomizing the humidification liquid, one or more rapidly vibrating mechanisms, or any combination of the preceding.

In an embodiment where atomizer 124 comprises piezoelectric ceramic discs, any suitable size of piezoelectric ceramic discs may be utilized. For example, the size of the piezoelectric ceramic discs may be altered in order to change the atomization effects (such as to change the particle size of the atomized humidification liquid) of the atomizer 124. In particular, a larger piezoelectric ceramic disc may be utilized in order to produce a larger particle size of the atomized humidification liquid. On the other hand, a smaller piezoelectric ceramic disc may be utilized in order produce a smaller particle size of the atomized humidification liquid. In particular embodiments, the size of the piezoelectric ceramic discs may be selected based on the type of surgical procedure to be conducted or based on the application of the atomized humidification liquid.

In particular embodiments, the piezoelectric ceramic disc may vibrate at any suitable frequency. For example, the piezoelectric ceramic disc may vibrate at 1.5 MHz. As another example, the piezoelectric ceramic disc may vibrate at a frequency greater than 1.5 MHz or at a frequency less than 1.5 MHz. According to the illustrated embodiment, the piezoelectric ceramic disc may be driven by a square wave. In particular embodiments, the amplitude (and thus the power) of the square wave may be altered in order to generate mist when needed. For example, the square wave may be altered automatically (by control module 178 or any suitable logic) in order to generate less mist, fog, or fine spray of the humidification liquid when less is needed, or to generate more mist, fog, or fine spray of the humidification liquid when more is needed. In particular embodiments, the amplitude of the square wave provided to the atomizer may be based on the flow rate of the gas entering humidification chamber 136. For example, if a low flow rate of gas is entering humidification chamber 136, the amplitude may be altered so that an amount of mist generated by atomizer matches the low flow rate of gas. Alternatively, if a high flow rate of gas is entering humidification chamber 136, the amplitude may be altered so that an amount of mist generated by atomizer matches the high flow rate of gas. In particular embodiments, the flow entering the humidification chamber 136 may be measured by a flow meter (such as an electronic flow meter or a mechanical flow meter) that communicates with the control module 178 or the insufflator 102. The flow meter may be included in the control housing 210 or the insufflator 102, or may be external to the control housing 210 or the insufflator 102.

In response to the square wave, the piezoelectric ceramic disc may contract and expand, thereby atomizing the humidification liquid. As such, the piezoelectric ceramic disc may create a mist, fog, or fine spray of the humidification liquid. In particular embodiments, the piezoelectric ceramic disc may utilize a “single side” in order to generate atomized humidification liquid. In particular embodiments, the piezoelectric ceramic disc may indirectly atomize the humidification liquid. For example, the vibrations of the piezoelectric ceramic disc may agitate an excitation liquid stored in humidification chamber 136 (as opposed to directly atomizing the humidification liquid, itself). The excitation liquid may be sterile water that is hermetically sealed within an excitation chamber 140 of the humidification chamber 136 (so as to prevent the excitation liquid from exiting the humidification chamber 136). Once the excitation liquid is agitated by the vibrations of the piezoelectric ceramic disc, the agitated excitation liquid may excite the humidification liquid also stored in humidification chamber 136. Such excitation of the humidification liquid by the excitation liquid (and the piezoelectric ceramic disc) may atomize the humidification liquid, resulting in a mist, fog, or fine spray of the humidification liquid.

In particular embodiments, atomizer 124 may be a reusable atomizer 124. For example, after a first surgical procedure, atomizer 124 may be uncoupled from humidification chamber 136 and reused in another surgical procedure with a different humidification chamber 136 and a different device 104. In another embodiment, each atomizer 124 may be a single use disposable atomizer 124. As such, atomizer 124 may be disposed after being used with a single patient.

In particular embodiments, atomizing (or nebulizing) the humidification liquid may greatly increase the likelihood that the compound that is atomized will remain a part of the humidified gas. For example, unlike traditional humidification elements (such as a hydrophilic retention media) which may retain crystalline portions of the humidification liquid (such as retaining the NaCl portion of a sterile saline solution), when the humidification liquid is vaporized by heating, the crystalline portion of the humidification liquid may be atomized and used to humidify the gas. As such, the invention may greatly increase the likelihood that the compound that is atomized will remain a part of the humidified gas.

Humidification chamber 136 may be any chamber operable to receive a gas from a source and humidify the received gas. According to the illustrated embodiment, humidification chamber 136 is a chamber that includes one or more openings 144 (opening 144a and opening 144b).

Openings 144 may be any opening in humidification chamber 136 that allows gas received from a source to enter humidification chamber 136. For example, according to the illustrated embodiment, after gas is filtered by filter 116 the gas may flow into humidification chamber 136 through openings 144. In particular embodiments, openings 144 may be positioned on humidification chamber 136 so as to generate a vortex flow 148 of the gas, as is discussed below. Although device 104 is described above as including two openings 144, device 104 may have any other number of openings 144. For example, device 104 may have more than two openings 144 (such as three openings 144, four openings 144, etc.) or less than two openings 144.

In particular embodiments, a cooling system (not shown) may be used in order to cool the humidification liquid contained in humidification chamber 136. In particular embodiments, such a cooling system may be utilized in order to insure that the humidification liquid's temperature does not surpass a given threshold. The cooling system may be any suitable cooling system. For example, the cooling system may be a cooling fan, a non-contacting heat exchanger using cold fluid surrounding the humidification chamber 136, any other cooling system, or any combination of the preceding. In particular embodiments, the cooling system may be part of control housing 210 of control module 178. As such, when device 104 is stored in, on, or near control housing 210, the cooling system may be utilized to cool the humidification liquid in humidification chamber 136. The cooling system may be any size and have any configuration. Furthermore, the size and configuration of the cooling system may be varied based on the application of device 104.

In particular embodiments where humidification chamber 136 indirectly atomizes the humidification liquid using, for example, an excitation liquid, humidification chamber may further include an excitation chamber (not shown). The excitation chamber refers to a chamber that contains the excitation liquid. For example, the excitation chamber may contain sterile water that excites the humidification liquid in humidification chamber 136. In particular embodiments, when the excitation liquid contained in the excitation chamber is agitated, the excitation liquid excites the humidification liquid (which may be located in a thin-membrane containment vessel (not shown) in humidification chamber 136), generating atomized humidification liquid in the form of a mist, fog, or fine spray.

As is discussed above, humidification chamber 136 humidifies the gas received from a source. According to the illustrated embodiment, in order to humidify the gas received from a source, humidification chamber 136 generates a vortex flow 148 of the received gas inside of the humidification chamber 136 and combines the vortex flow 148 of the received gas with at least a portion of the atomized humidification liquid. A vortex flow 148 (or vortex dispersion) may refer to a circular, spiral, or helical motion of the gas that attracts the gas towards the wall of the humidification chamber 136. The vortex flow 148 may result in an outward deflection of the gas, a radial dispersal of the gas, and/or a centrifugal spinning action of the gas inside of the humidification chamber 136.

In particular embodiments, the vortex flow 148 of the gas may pull the atomized humidification liquid towards delivery conduit 152, thereby creating a space that may allow additional atomized humidification liquid to be generated by atomizer 124. Such a vortex flow 148 may provide one or more advantages over traditional atomization systems. For example, an atomized humidification liquid may traditionally be provided to a standard flow of gas (as opposed to a vortex flow 148). Unfortunately, a standard flow of gas may not be able to pull the atomized humidification liquid away from the atomizer. In particular, in a pressurized system (such as occurs in endoscopic procedures), the pressure inside of the humidification chamber may force the atomized humidification liquid towards the bottom of the humidification chamber (e.g., back to the atomizer). As such, because the atomized humidification liquid is forced to the bottom of the humidification chamber, it may not be properly combined with the flow of gas, thus preventing the gas from being properly conditioned. To the contrary, in certain embodiments, a vortex flow 148 of the gas may pull the atomized humidification liquid towards the top of the humidification chamber 136, allowing the gas to be adequately and efficiently conditioned. The vortex flow 148 of gas may be generated by the humidification chamber 136 in any manner. As a first example, the vortex flow 148 may be generated based on openings 144. In particular, the openings 144 may be off-centered from each other and/or may direct the flow of gas into humidification chamber 136 at an angle that causes the gas to flow upwards along the inner circumference of the humidification chamber 136. For instance, each of the openings 144 may direct the gas into the humidification chamber 136 at a 45° angle towards the top of the humidification chamber 136 and also at a 45° angle towards the inside circumference of the humidification chamber 136. As such, the flow of gas may generate a vortex flow 148. Although the above example has been described as directing the gas at a 45° angle towards the top of the humidification chamber 136 and also at a 45° angle towards the inside circumference of the humidification chamber 136, in particular embodiments, any other angles may be utilized to generate the vortex flow 148. For example, openings 144 may direct the gas into the humidification chamber at a 20° angle, 25° angle, 30° angle, 35° angle, 40° angle, 50° angle, 55° angle, 60° angle, or any other degree of angle towards the top of the humidification chamber and also at a 20° angle, 25° angle, 30° angle, 35° angle, 40° angle, 50° angle, 55° angle, 60° angle, or any other degree of angle towards the inside circumference of the humidification chamber 136 provided that a vortex is generated. Furthermore, openings 144 may direct the gas at a different angle towards the top of the humidification chamber 136 than the angle utilized to direct the gas towards the inside circumference of the humidification chamber. For instance, the openings 144 may direct the gas into the humidification chamber at a 45° angle towards the top of the humidification chamber 136 and also at a 35° angle towards the inside circumference of the humidification chamber 136. Additionally, one or more of the openings 144 may direct the flow of gas into the humidification chamber 136 at different angles than the other openings 144. As another option, one or more of the openings 144 might just direct the flow towards the top of the humidification chamber 136 or towards the inside circumference.

Although the vortex flow 148 of the gas has been described as being generated based on openings 144 of humidification chamber 136, the vortex flow 148 of the gas may be generated in any other manner. For example, the vortex flow 148 of the gas may be generated by causing the gas to enter the humidification chamber 136 through one or more beveled openings 144, or by causing the gas to pass over spiraled (or rifled) areas positioned inside of the humidification chamber 136. Furthermore, any other manner of generating the vortex flow 148 may be utilized.

In particular embodiments, humidification chamber 136 may further include a impeller device (not shown) located near the top of humidification chamber 136. The impeller device may allow atomized gas to exit the humidification chamber 136 towards delivery conduit 152 in an appropriate spin direction. The impeller device may further prevent (or reduce the amount of) large particles of humidification liquid from exiting the humidification chamber 136. As such, the impeller device may act as a shield against humidification liquid particles that may be larger than desired, preventing them from exiting the humidification chamber 136 towards the delivery conduit 152. The impeller may have any suitable shape, and may be made of any suitable non-porous material. The impeller may be a static impeller or a dynamic impeller (e.g., the impeller may be able to move, such as, for example, by spinning) Furthermore, the dynamic impeller may be a powered dynamic impeller (e.g., powered from an electrical source or any other power source) or an unpowered dynamic impeller (e.g., where the motion may be caused by the flow of gas in the humidification chamber 136). In particular embodiments, humidification chamber 136 may not include an impeller.

Humidification chamber 136 may humidify the gas to any particular relative humidity. For example, the gas may be humidified so that it is within a range of relative humidity at the exit of the device 104 for delivery adjacent to or into the patient. It may also be within any of the following humidity ranges as the gas enters the patient through the exit of a delivery device. The relative humidity level may be above 40%, above 50%, above 60%, above 70%, above 75%, above 80%, above 85%, or above 90% relative humidity. In further embodiments, the range of relative humidity may be between 65-80%, between 70-85%, between 75-90%, between 80-95%, or any other suitable range. In particular embodiments, the relative humidity of the gas does not always need to be within a particular range of relative humidity. For example, changes in the flow conditions or other influences may cause the relative humidity of the gas to be outside of the range of relative humidity for a period of time. In some embodiments, the relative humidity may be between 95% and 100%. In one embodiment, humidification chamber 136 may humidify the gas to a predetermined (or preselected) range of relative humidity. For example, a user may select a range of relative humidity using the control module 178, and the control module 178 may cause the humidification chamber 136 to generate a particular amount of atomized humidification liquid in order for the relative humidity of the gas to be within the selected range. Furthermore, although device 104 of FIGS. 1 and 2 has been described as being capable of humidifying the gas to any of the above described humidity levels when device 104 includes the components described above, device 104 may also be able to humidify the gas to any of the above described humidity levels when it includes any combination of any of the described components and any of the options or features described herein, as would be understood by one of ordinary skill in the art.

In particular embodiments, the amount of humidity in the gas may be controlled by control module 178. For example, the control module 178 may vary the pulsing frequency (or duty cycle) of the atomizer 124 in order to control the amount of humidity in the gas. In such an example, the control module 178 may increase the pulsing frequency (or duty cycle) of the atomizer 124 in order to increase the relative humidity, or decrease the pulsing frequency (or duty cycle) of the atomizer 124 in order to decrease the relative humidity. The increase or decrease of the pulsing frequency (or duty cycle) of the atomizer 124 may be based on the measured flow rate of the gas entering the humidification chamber 136 and/or the selection (or setting) of the relative humidity. As another example, the amount of power administered to atomizer 124 may be varied in order to control the amount of humidity in the gas.

The control module 178 may include relative humidity profiles (or any other power/humidity profiles) that allow the control module 178 to determine the rate of pulsing (or the duty cycle or the amount of power) that is needed to provide a range of relative humidity for a particular flow rate of the gas. As such, the control module 178 may not need to measure the relative humidity of the gas. Instead, the control module 178 may determine the flow rate of the gas entering the humidification chamber 136 and then calculate (based on the relative humidity profiles) the rate of pulsing (or the duty cycle or the amount of power) that is needed to provide the range of relative humidity for the measured flow rate of the gas.

As another example, the control module 178 may control (or check its relative humidity profile calculations) based on feedback obtained from a relative humidity sensor (not shown). In one embodiment, the relative humidity sensor may be provided anywhere within the flow of gas. For example, the relative humidity sensor may be positioned in the flow path of gas exiting the humidification chamber 136. As another example, the relative humidity sensor may be present in the delivery conduit 152 at the most proximal portion of the fluid of the delivery conduit 152 (relative to the patient) to ensure that the gas stream is conditioned to the appropriate levels prior to being delivered adjacent to or into the patient. In a further embodiment, the relative humidity sensor may be provided in any other location that allows it to sense the relative humidity of the gas. The relative humidity sensor may be a humidity-sensitive capacitor sensor, such as a capacitive humidity sensor manufactured by Philips Corporation, which changes capacitance in response to humidity changes. Other humidity sensors can also be used. The humidity sensor measures the relative humidity of the gas as it passes through the delivery conduit 152 to enable monitoring of the gas humidity, and in order to provide an indication of the amount of liquid remaining in the humidification chamber 136. In one embodiment, if the relative humidity of the gas falls below a predetermined relative humidity threshold, a signal may alert a user about the drop in relative humidity. In such an embodiment, an additional amount of the humidification liquid may be injected into the humidification chamber 136. For example, the humidification liquid may be injected into the humidification chamber 136, as is discussed in FIG. 1 As such, in one embodiment, the additional amount of the humidification liquid may increase the relative humidity of the gas.

In particular embodiments, in order to provide a relatively constant rate of atomization (or nebulization), a consistent level of humidification liquid may be maintained in humidification chamber 136. In order to maintain such a consistent level of humidification liquid, the level of humidification liquid stored in humidification chamber 136 may be recharged. In particular embodiments, any apparatus or device may be utilized to recharge humidification chamber 136. For example, as is discussed above, the humidification liquid stored in humidification chamber 136 may be recharged using syringe 200.

As another example, the humidification liquid in the humidification chamber may be recharged using other devices, such as an Intravenous (IV) bag (or Saline bag) controlled by a pressurized pump system or pressure cuff, a metering system (such as a metered syringe), a peristaltic pump, an active filling system, any automatic charging system, or any other suitable device. In such examples, the control module 178 may control the amount of humidification liquid that is provided by these devices so that the control module 178 may maintain a relatively consistent level of humidification liquid in the humidification chamber 136. The control of these devices by control module 178 may be based on the measured flow rate of the gas entering the humidification chamber 136 and the relative humidity profiles (or the measured relative humidity). In further embodiments, the control of these device by control module 178 may be based on measurements of the fill level of the humidification liquid in humidification chamber 136. In such embodiments, the fill level may be measured and provided to the control module 178 by optical sensors, resistance sensors, load cells, capacitive sensors, and/or inductive sensors in humidification chamber 136. These sensors may directly measure the fill level of the humidification liquid or may indirectly measure the fill level, such as by measuring the amount of power being utilized by atomizer 124, the amount of work conducted by atomizer 124, or the pulsing frequency (or duty cycle) of the atomizer 124.

As a further example, the humidification liquid in humidification chamber 136 may be recharged using a float system. For example, humidification chamber 136 may include a preliminary fill chamber that feeds humidification liquid into humidification chamber 136 in order to maintain a consistent level of the humidification liquid. In such an example, as humidification liquid is atomized (or nebulized), the level of humidification liquid in humidification chamber 136 may decrease, thereby causing the float system to fall with the level of the humidification liquid. When the float system falls, the float system may unblock a port that connects the humidification chamber 136 to the preliminary fill chamber. Thus, humidification liquid may flow into the humidification chamber 136 until the level of the humidification liquid rises, causing the float system to once again block the port. In particular embodiments, this cycle may continue continuously in order to maintain a consistent level of humidification liquid in humidification chamber 136.

As a further example, the humidification liquid in humidification chamber 136 may be recharged using an atomization measuring system. In such an example, the amount of power being utilized by atomizer 124, the amount of work conducted by atomizer 124, or the pulsing frequency (or duty cycle) of the atomizer 124 may be measured. Once a predetermined amount of power has been utilized or a predetermined amount of work has been conducted, atomization measuring system may cause a preliminary fill chamber (discussed above) to supply humidification liquid to humidification chamber 136. Therefore, the amount of humidification liquid in humidification chamber 136 may be correlated with the amount of power being utilized by atomizer 124, the amount of work conducted by atomizer 124, or the pulsing frequency (or duty cycle) of the atomizer 124.

In particular embodiments, the medicant provided to the humidification chamber (or the humidification liquid) may be recharged. The recharge of the medicant may be accomplished using any of the apparatuses, devices, and/or methods discussed above with regard to recharging the humidification liquid. Furthermore, the recharge of the medicant may be controlled independently of the recharge of the humidification liquid. For example, while a consistent level of humidification liquid may be maintained (such as by control module 178) in the humidification chamber 136, the amount of medicant provided into the humidification chamber 136 (or into the humidification liquid provided into the humidification chamber 136) may be varied throughout the procedure. The independent control of the medicant recharge may be performed by the control module 178. As an example, such independent control may be performed in accordance with one or more medicant profiles and/or a selection by a user.

Delivery conduit 152 may be any conduit that receives humidified gas from the humidification chamber 136 (as discussed below) and provides the humidified gas adjacent to or into a patient. Delivery conduit 152 may be flexible and may have any suitable length (such as, for example, any length between 12 inches and 12 feet). In one embodiment, delivery conduit 152 may be sufficiently long to permit the humidification chamber 136 to be placed within 12 inches of the patient. Delivery conduit 152 may comprise any material. For example, delivery conduit may comprises polyvinyl chloride (PVC).

According to the illustrated embodiment, in addition to providing the humidified gas adjacent to or into a patient, delivery conduit 152 may be further operable to warm at least a portion of the humidified gas using heating elements 156 (e.g., heating element 156a and heating element 156b). Heating elements 156 may warm the humidified gas to any particular temperature. For example, the temperature of the gas may be warmed so that it is within any of the following temperature ranges as it exits the delivery conduit 152 of device 104 for delivery adjacent to or into the patient. It may also be within any of the following temperature ranges as the gas enters the patient through the exit of a delivery device. In one embodiment, the temperature range that the gas is warmed to is approximately 35°-40° C. For example, the gas may be warmed using a predetermined temperature set point, such as, 37° C. Other set points could be used without departing from the scope of the invention. Warming to a set point may result in a temperature range at the exit of the device 104. In another embodiment, the temperature that the gas is warmed to may be below 35° C. In a further embodiment, the temperature that the gas is warmed to may be above 40° C. In particular embodiments, the temperature range that the gas is warmed to may be approximately 28°-33° C., 30°-35° C., 32°-37° C., 37°-42° C., 39°-44° C., 30°-45° C., 20°-45° C., or any other suitable temperature range. In particular embodiments, the gas does not always need be warmed to a particular temperature range. For example, changes in the flow conditions or other influences may cause the temperature of the gas to be outside of the temperature range for a period of time. In some embodiments, the temperature may be adjustable and in others it may not. For example, the temperature may be adjustable based on the type of procedure device 104 is used for. This may allow for a different temperature when device 104 is used for, as an example, inhalation purposes. As another example, the temperature may be adjustable based on the type of patient. This may allow a different temperature when device 104 is used on, as an example, pediatric patients. As a further example, the temperature may be selected (and adjustable) by a user. In such an example, the user may select any temperature, or adjust the temperature to any temperature.

In particular embodiments, by warming the humidified gas, heating elements 156 may vaporize at least a portion of the atomized humidification liquid in the humidified gas prior to the humidified gas being provided adjacent to or into the patient. Vaporization of the atomized humidification liquid may refer to transitioning the humidification liquid from a liquid phase to a gas phase. Such vaporization (or partial vaporization) may both alter the temperature of the gas received adjacent to or in the patient, and may also reduce potential visualization issues that could arise from the atomized humidification liquid, as is discussed below.

A heating element 156 may be any heating element. For example, heating element 156 may be a resistive type heating wire, such as a coiled and coated resistive type heating wire constructed from nickel-chromium. In such an example, the coating may take the form of any flexible, biocompatible plastic-polymer based compound, such as Polytetrafluoroethylene (PTFE or Teflon®), PEBAX®, any coating used in catheters, or any other suitable coating. Heating element 156 may have any configuration. For example, heating element 156 may be formed in a spiral configuration, a straight configuration, any other configuration, or any combination of the preceding. Heating element 156 may be located anywhere in delivery conduit 152. Furthermore, the position of heating element 156 (in relation to the proximity to the patient) may be altered depending on the specific application of device 104 and the type of gas to be warmed.

In particular embodiments, heating element 156 may be a resistive type heating element that is co-extruded inside a wall of delivery conduit 152. For example, heating element 156 may be co-extruded in the luminary walls of delivery conduit 152. In such an example, heating element 156 may be drawn through the extrusion die and frozen inside the wall of a polymer delivery conduit 152 during the extrusion process. Any number of heating elements 156 could be drawn into the walls of delivery conduit 152. Furthermore, after extrusion, delivery conduit 152 may be cut, exposing the heating element 156 for coupling to a communication cable, thereby allowing control module 178 to alter the power transmitted to heating element 156.

According to the illustrated embodiment, delivery conduit 152 includes two heating elements 156 (heating element 156a and heating element 156b). Heating element 156b is operable to vaporize the atomized humidification liquid. Heating element 156b may have any temperature and any length for vaporizing the atomized humidification liquid. As an example, heating element 156b may have a temperature of 50-55° C. and a length of approximately 5 feet long. Heating element 156a is operable to cause the vaporized humidification liquid and the gas to exit the delivery conduit 152 and/or enter the patient at a particular temperature range. For example, heating element 156a may cause the vaporized humidification liquid and the gas to exit the delivery conduit 152 and/or enter the patient at approximately 20° -45° C. As other example, heating element 156a may cause the vaporized humidification liquid and the gas to exit the delivery conduit 152 and/or enter the patient at any other temperature range, as is discussed above. In particular embodiments, heating element 156a may further provide heat to vaporized humidification liquid in order to keep the vaporized humidification liquid vaporized. As such, heating element 156a may cause the vaporized humidification liquid and the gas to exit the delivery conduit 152 and/or enter the patient at a particular temperature range, while also causing the vaporized humidification liquid to remain vaporized. Heating element 156a may have any temperature and any length. As an example, heating element 156a may have a length of approximately 5 feet long. As another example, the temperature of heating element 156a may be varied based on the flow rate of the gas. For example, heating element 156a may have a lower temperature when the gas has a high flow rate, and a lower temperature when the gas has a lower flow rate. In particular embodiments, if the flow rate of the gas is high enough, heating element 156a may not need to be turned on at all (or turned on only minimally).

Although delivery conduit 152 has been illustrated as including two heating elements 156, in particular embodiments, delivery conduit 152 may include more than two heating elements 156 (such as three) or less than two heating elements 156 (such as one). In an embodiment where delivery conduit 152 includes only one heating element 156, the heating element may have multiple zones. In particular embodiments, such zones may allow each zone to be heated to a different temperature, or may allow only a subset of the zones to be heated at a particular time. For example, when gas is flowing through delivery conduit 152 at a lower flow rate, the gas may be heated at a zone that is closer to humidification chamber 136. This may allow for the vaporization of the atomized humidification liquid to occur and also may allow the gas to fall back to a desired temperature prior to being delivered adjacent to or into the patient. On the other hand, if the gas is flowing through the delivery conduit 152 at a high flow rate, the gas may be warmed using a zone that is closer to the patient. Furthermore, although heating element 156 has been described above as including multiple zones, in further embodiments, an actively placed heating element 156 may be utilized in addition to or instead of the multiple zones of heating element 156.

Although delivery conduit 152 has been illustrated as including a heating element 156b operable to vaporize the atomized humidification liquid, in particular embodiments, the heating element 156b may not be used (or may not be used at a temperature that vaporizes atomized humidification liquid). As such, atomized humidification liquid (as opposed to vaporized humidification liquid) may be provided into or adjacent to the patient. Furthermore, in particular embodiments, delivery conduit 152 may not include a heating element operable to vaporize the atomized liquid.

In particular embodiments, the temperature of heating elements 156 (and thereby the warming of the gas provided by heating elements 156) may be controlled by control module 178. In such embodiments, the amount of power administered to heating elements 156 (or the amount of power independently controlled and administered to each of heating element 156a and heating element 156b) may be varied based on feedback obtained from one or more temperature sensors (not shown). In one embodiment, the temperature sensors may be provided anywhere within the flow of gas. For example, a temperature sensor may be present in the delivery conduit 152 at the most proximal portion of the fluid of the delivery conduit 152 (relative to the patient) to ensure that the gas stream is conditioned to the appropriate levels prior to being delivered adjacent to or into the patient. In a further embodiment, a temperature sensor may be provided in any other location that allows it to sense the temperature of the gas. For example, a temperature sensor may be provided immediately after heating element 156b, so that the temperature of the vaporized humidification liquid may be measured. In one embodiment, the temperature sensor is a thermistor. In another embodiment, the temperature sensors are resistance temperature detectors (RTD). In one embodiment, the temperature sensors may be accurate to within about 0.2° C. In an alternative embodiment, temperature of the gas can be sensed indirectly by sensing the temperature of the heater. Infrared sensors could also be used.

In particular embodiments, vaporizing (and/or warming) the atomized humidification liquid in the delivery conduit 152 may provide one or more advantages over heating elements used in traditional systems. For example, traditionally, an atomized humidification liquid may be vaporized before the gas (which has been combined with the atomized humidification liquid) enters the delivery conduit. Unfortunately, when the humidification liquid includes medicants, vaporization of the atomized humidification liquid may cause the medicants to drop out of the gas and drop back towards the bottom of the humidification chamber. As such, the gas may not be conditioned with adequate amounts of the medicants. In particular embodiments, by vaporizing (either completely or partially) an atomized humidification liquid after the gas (which includes the atomized humidification liquid) enters the delivery conduit 152, the amount of medicant dropping out of the gas may be reduced. Furthermore, even if the medicant does drop out of the gas, it may not occur until after the medicant is already in the delivery conduit 152. As such, the force of the gas may push the medicant towards the patient, as opposed to allowing it to flow back into the humidification chamber 136.

In particular embodiments, vaporizing (either completely or partially) an atomized humidification liquid may further prevent the fluid from obstructing the vision of the surgeon performing the medical procedure (or decrease the amount of such obstruction). For example, although the atomized particles may be big enough to obstruct the vision of the surgeon, the vaporization of these atomized particles may reduce the particle size, thereby preventing the fluid from obstructing the vision of the surgeon performing the medical procedure (or decreasing the amount of such obstruction).

Furthermore, vaporizing (and/or warming) the atomized humidification liquid in the delivery conduit 152 may allow the vaporization and the humidification of the gas to be controlled independently. In particular embodiments, this may provide for an optimum balance between heating and humidifying the gas. For example, the independent control may prevent a super-saturated peritoneal environment that can lead to lens fogging, or condensation that collects on the walls of the laparoscope and that may eventually migrate down the scopes' shaft and occlude the view of the lens.

Modifications, additions, or omissions may be made to the device 104 without departing from the scope of the invention. The components of the device 104 may be integrated or separated. Moreover, the operations of the device 104 may be performed by more, fewer, or other components. For example, the operations of the humidification chamber 136 may be performed by more than one component.

Although FIGS. 1 and 2 have been described above as including particular components, the systems of FIGS. 1 and 2 may include any combination of any of the described components and any of the options or features described herein, as would be understood by one of ordinary skill in the art. For example, any of the options or features described herein may be utilized in combination with the illustrated embodiments of FIGS. 1 and 2 and/or any number of the other options or features also described herein, as would be understood by one of ordinary skill in the art.

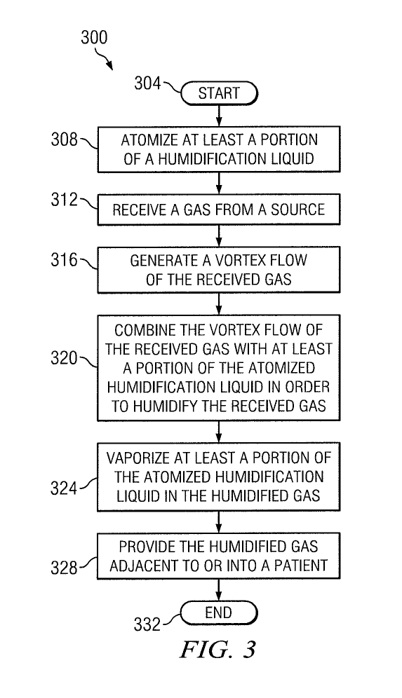

FIG. 3 illustrates one embodiment of a method 300 for performing a surgical procedure. In particular embodiments, one or more steps of method 300 may be performed using one or more components of FIGS. 1 and 2 and all of the options discussed above.

At step 304 the method begins. In particular embodiments, in order for the method 300 to begin, device 104 may be setup for operation, as is described below. The AC/DC converter 180 is plugged into a 110 V AC (or any other power standard in any country, such as 220 V AC, 240 V AC, etc.) power source, such as a wall outlet or a power strip. The control module 178 is connected to the AC/DC converter 180. In another embodiment, the device 104 may be powered by a battery or photovoltaic source. The tubing set is then installed by attaching one end of the tube segment 112 to the outlet of the control housing 210 (which receives gas from insufflator 102 and, based on control module 178, provides the gas to the outlet) by the Luer lock 108. In a further embodiment, the insufflator 102 may include control module 178 (as opposed to control housing 210) and may be able to perform all of the functions discussed herein with regard to control module 178. In such an embodiment, tube segment 112 may be attached directly to the outlet of insufflator 102 by the Luer lock 108. The tube segment 112, tube segment 120, and delivery conduit 152 may be pre-attached to the filter 116 and the humidification chamber 136 for commercial distribution of the device 104. The cable 170 is installed into the control housing 210 by the connector 172. The humidification chamber 136 is charged with a supply of humidification liquid by the syringe 200. For example, 50 cc of a humidification liquid, such as sterile water or saline, is drawn into the syringe 200. The syringe 200 is then inserted into the charging port 190 so that a needle or cannula of the syringe 200 penetrates the resealable member and the humidification liquid is injected into the humidification chamber 136. The syringe 200 is then removed from the charging port 190, and the charging port 190 seals itself.

The syringe 200 may then be re-filled and placed in an automatic pump (such as a syringe pump) controlled by control module 178. Control module 178 may then maintain a consistent level of humidification liquid in the humidification chamber 136. In addition, another syringe (or any other delivery device) may also be connected to device 104 for providing a medicant to humidification chamber 136. In particular embodiments, control module 178 may independently control the amount of humidification liquid provided into humidification chamber 136 and the amount of medicant provided into humidification chamber 136. The amount of medicant provided into the humidification chamber 136 may be based on one or more medicant profiles, as is discussed above. The free end of the delivery conduit 152 is attached to a gas delivery device, such as a trocar, verres needle, endoscope, or a tube that enters a body cavity or space that delivers the filtered, heated, and/or humidified gas adjacent to or into the body of a patient, by the Luer lock 168 or other appropriate connector. In another embodiment, the humidification chamber 136 may be precharged with liquid, thus not requiring a charge prior to operation.

Once device 104 is setup for operation, the method moves to step 308, where at least a portion of a humidification liquid is atomized. In particular embodiments, the portion of the humidification liquid may be atomized by atomizer 124. As such, atomizer 124 may generate atomized humidification liquid, such as in the form of a mist, fog, or fine spray.

At step 312, a gas is received from a source. In one embodiment, the gas may be received from insufflator 102. For example, once the insufflator 102 is activated, it receives gas from a gas supply cylinder and regulates the pressure and flow rate of the gas, both of which can be adjusted by the operator. The pressure and volumetric flow rate are controlled by adjusting controls (not shown) on the insufflator 102. Insufflator gas then flows into control housing 210, where control module 178 can regulate the amount of gas provided to device 104, the pressure of the gas, the flow rate of the gas, the temperature of the gas, or any other property of the gas. In particular embodiments, the properties of the gas may be selected by a user. For example, the user may program the control module 178 to provide gas having the particular properties selected by the user (e.g., a particular flow rate, etc.). Based on control module 178, control housing 210 may provide the gas through the tube segment 112 into the optional filter 116 where it is filtered, and then through tube segment 120 into humidification chamber 136, such as through openings 144.

At step 316, a vortex flow of the received gas is generated. In particular embodiments, humidification chamber 136 may generate the vortex flow 148 in any manner. For example, as is discussed above, the vortex flow 148 may be generated based on openings 144 (which may be off-centered from each other and/or may direct the flow of gas into humidification chamber 136 at an angle that causes the gas to flow upwards along the inner circumference of the humidification chamber 136), by directing the gas through one or more beveled openings 144, by causing the gas to pass over spiraled (or rifled) areas positioned inside of the humidification chamber 136, any other manner, or any suitable combination of the preceding.

At step 320, the vortex flow of the received gas is combined with at least a portion of the atomized humidification liquid in order to humidify the received gas. In one embodiment, such humidification of the gas may occur in the humidification chamber 136. For example, by generating the vortex flow 148, the vortex flow 148 may combine with the atomized humidification liquid (such as atomized humidification liquid in the form of a mist, fog, or fine spray) and pull the atomized humidification liquid away from atomizer 124. In particular embodiments, this may humidify the received gas, and allow atomizer 124 to generate more atomized humidification liquid.

Humidification chamber 136 may humidify the received gas such that the gas exiting humidification chamber 136 is within a predetermined (or preselected) range of relative humidity. In one embodiment, the gas may be humidified so that it is within a range of relative humidity at the exit of the device 104 for delivery adjacent to or into the patient. It may also be within any of the following humidity ranges as the gas enters the patient or the area adjacent to the patient through the exit of a delivery device. The relative humidity level may be above 40%, above 50%, above 60%, above 70%, above 75%, above 80%, above 85%, or above 90% relative humidity. In further embodiments, the range of relative humidity may be between 65-80%, between 70-85%, between 75-90%, between 80-95%, or any other suitable range. In some embodiments, the relative humidity may be between 95% and 100%. In particular embodiments, the range of relative humidity of the gas may be selected by a user. For example, the user may program the control module 178 to maintain the relative humidity of the gas within a particular range. In such an example, the user may program the control module 178 with any range of relative humidity.

In one embodiment, if the device 104 is operated with the humidification chamber 136 not charged with humidification liquid either because the user forgot to manually charge it before initiating operation, or the device 104 was sold without a pre-charge of humidification liquid (e.g., in a dry state), the relative humidity of the gas will be detected (by a relative humidity sensor, for example) to be below the predetermined threshold and the alarm will be activated, alerting the user that the humidification chamber 136 requires charging of humidification liquid. In one embodiment, the device 104 will automatically issue an alarm to alert a user to the need for charging the humidification chamber 136 with humidification liquid, thereby avoiding further delivery of unhumidified gas adjacent to or into the patient. In another embodiment, the control module 178 may detect the absence of humidification liquid in the humidification chamber 136 (such as by a relative humidity sensor or by measuring the amount of humidification liquid in the humidification chamber, as is discussed above), and may cause the humidification chamber 136 to be re-charged with humidification chamber 136. For example, the control module 178 may communicate with an automatic pump (such as a syringe pump, or any other device) controlled by control module 178. The automatic pump may then re-charge the humidification chamber 136. In particular embodiments, if the automatic pump (or other re-charging device) does not contain humidification liquid (or if the amount of humidification liquid is low), an alarm may be activated, alerting the user that the automatic pump (or other re-charging device) needs to be re-filled or replaced.

At step 324, at least a portion of the atomized humidification liquid in the humidified gas is vaporized. In particular embodiments, heating element 156 in delivery conduit 152 may vaporize the atomized humidification liquid. In particular embodiments, such vaporization may cause the humidified gas exiting device 104 to be within a desirable physiological temperature range (for example, 35° to 40° C., though any desired temperature range can be predetermined (or preselected), as is discussed above).

At step 328, the humidified gas is provided adjacent to or into a patient. In particular embodiments, the humidified gas may be provided adjacent to or into the patient by the delivery conduit 152 and/or a gas delivery device, such as a trocar, verres needle, endoscope, a mouthpiece, a wound bandage delivery system, or a tube that enters a body cavity or space that delivers the filtered, warmed, and/or humidified gas adjacent to or into the body of a patient. In particular embodiments of method 300, the atomized humidification liquid may not be vaporized (or even warmed). In such embodiments, only humidified gas may be provided adjacent to or into the patient. After the humidified gas is provided adjacent to or into the patient, the method moves to step 332, where the method ends.

Although the method 300 has been described above as ending after the humidified gas is provided adjacent to or into the patent, in particular embodiments, the method may include further steps, as is discussed below. For example, the relative humidity and temperature of the gas exiting device 104 may be monitored. The control module 178 may monitor the relative humidity of the gas exiting the humidification chamber 136 (or device 104) and further regulate the temperature of the gas exiting device 104. In particular, a microcontroller may generate a recharge signal when the relative humidity of the gas in the humidification chamber 136 drops below the predetermined relative humidity threshold, indicating that the humidification liquid supply in the humidification chamber 136 requires replenishing. As another example, a microcontroller may generate a recharge signal when the level of humidification liquid in the humidification chamber 136 has dropped below a particular level (or if it is not being maintained at a consistent level), indicating that an automatic re-charging system (such an automatic pump) should re-charge the humidification chamber. Furthermore, an audible alarm may be issued by a buzzer and/or a visual alarm may be issued by an LED to warn the medical attendant or user that the humidification chamber 136 requires recharging or that the automatic re-charging system needs to be re-filled or replaced. In one embodiment, the microcontroller may continue the alarm until the humidity in the humidification chamber 136 returns to a level above the predetermined relative humidity threshold or the level of the humidification liquid in the humidification chamber 136 becomes consistent again, which will occur when the humidification chamber 136 is recharged with humidification liquid. Moreover, the microcontroller may issue a second alarm, such as by energizing a LED, when the relative humidity level of gas in the humidification chamber 136 drops below a critical relative humidity threshold or when the amount of the humidification liquid in the humidification chamber drops below a critical fill level, at which point electrical power to the humidification chamber may be terminated. In a further embodiment, the microcontroller may control the temperature of the gas by controlling electrical power supplied to the heating element 156.

As another example, method 300 may further include a filtration step. For example, after the humidified gas has entered the patient, the method may include filtering out at least a portion of the medicant in the gas. The medicant that is filtered out may be any medicant that is not absorbed by the body of the patient. In particular embodiments, the medicant may be filtered out using any suitable filtration method. As an example, a filter (such as a filtration trocar) attached to a removal system (such as a vacuum or any other removal system) may be inserted into the patient. In such an example, once the procedure is completed or once the medicant is no longer needed in the body, additional gas (such as additional humidified and/or warmed gas) that does not include that particular medicant (e.g., it may include other medicant types) may be provided into or adjacent to the patient, resulting in the additional gas entering the patient. This additional gas may flush the medicant out of the body through the filter. In particular embodiments, such a filtration step may allow for the removal of medicants from the body, and may further allow medicants to be safely removed from the body without exposing the procedure site or the users from contact with the medicant (which could possibly be dangerous, such as a chemotherapy agent).

Modifications, additions, or omissions may be made to method 300. For example, one or more steps in method 300 of FIG. 3 may be performed in parallel or in any suitable order. Furthermore, any other components may be utilized to perform one or more steps in method 300 of FIG. 3.

Although the present invention has been described with several embodiments, a myriad of changes, variations, alterations, transformations, and modifications may be suggested to one skilled in the art, and it is intended that the present invention encompass such changes, variations, alterations, transformations, and modifications as fall within the scope of the appended claims.

What is claimed is:

1. A system for performing a surgical procedure, comprising:
an atomizer operable to atomize at least a portion of a humidification liquid in a humidification chamber;
the humidification chamber operable to:
receive a gas from a source;
generate a vortex flow of the received gas inside of the humidification chamber; and
combine the vortex flow of the received gas with at least a portion of the atomized humidification liquid in order to humidify the received gas; and
a delivery conduit operable to:
receive the humidified gas from the humidification chamber; and
provide the humidified gas adjacent to or into a patient.

2. The system of claim 1, wherein the delivery conduit comprises a heating element operable to warm at least a portion of the humidified gas prior to the humidified gas being provided adjacent to or into the patient.

3. The system of claim 2, wherein the heating element operable to warm at least the portion of the humidified gas prior to the humidified gas being provided adjacent to or into the patient comprises the heating element operable to vaporize at least a portion of the atomized humidification liquid in the humidified gas prior to the humidified gas being provided adjacent to or into the patient.

4. The system of claim 3, wherein the heating element comprises a resistive type heating wire.

5. The system of claim 4, wherein at least a portion of the resistive type heating element is coextruded inside a wall of the delivery conduit.

6. The system of claim 1, wherein the humidification chamber comprises a plurality of openings positioned so as to generate the vortex flow of the received gas inside of the humidification chamber.

7. The system of claim 1, wherein the humidification chamber comprises one or more spiraled portions positioned as to generate the vortex flow of the received gas inside of the humidification chamber.

8. The system of claim 1, wherein the atomizer comprises one or more piezoelectric elements.

9. The system of claim 8, wherein the one or more piezoelectric elements comprise one or more piezoelectric ceramic discs.

10. The system of claim 1, wherein the atomizer comprises one or more pneumatic pumps.

11. The system of claim 1, wherein the humidification liquid comprises sterile water or saline.

12. The system of claim 11, wherein the humidification liquid further comprises one or more medicants.

13. The system of claim 12, wherein the one or more medicants are selected from a group consisting of:
one or more one or more anesthetics;
one or more anticoagulants;
one or more antihypertensives;
one or more antibiotics;
one or more anti-adhesive agents; and
one or more chemotherapeutic agents.

14. The system of claim 1, wherein the humidification chamber operable to combine the vortex flow of the received gas with at least the portion of the atomized humidification liquid in order to humidify the received gas comprises the humidification chamber operable to combine the vortex flow of the received gas with at least the portion of the atomized humidification liquid in order increase the humidity of the received gas to a range of 80% through 95% relative humidity.

15. The system of claim 1, wherein the humidification chamber operable to combine the vortex flow of the received gas with at least the portion of the atomized humidification liquid in order to humidify the received gas comprises the humidification chamber operable to combine the vortex flow of the received gas with at least the portion of the atomized humidification liquid in order increase the humidity of the received gas to above 70% relative humidity.

16. The system of claim 1, wherein the humidification chamber operable to combine the vortex flow of the received gas with at least the portion of the atomized humidification liquid in order to humidify the received gas comprises the humidification chamber operable to combine the vortex flow of the received gas with at least the portion of the atomized humidification liquid in order increase the humidity of the received gas to above 95% relative humidity.

17. The system of claim 1, wherein the gas comprises carbon dioxide.

18. The system of claim 1, further comprising a filter operable to filter the gas prior to the gas being received by the humidification chamber.

19. The system of claim 1, further comprising a gas delivery device coupled to the delivery conduit, wherein the gas delivery device is selected from a group consisting of:
a trocar;
a verres needle;
an endoscope;
a diffusing gas delivery device;
a mouthpiece; and
a tube that is adjacent to or enters the patient.

20. A method for performing a surgical procedure, comprising:
atomizing at least a portion of a humidification liquid;
receiving a gas from a source;
generating a vortex flow of the received gas;
combining the vortex flow of the received gas with at least a portion of the atomized humidification liquid in order to humidify the received gas; and
providing the humidified gas adjacent to or into a patient.

21. The method of claim 20, further comprising vaporizing at least a portion of the atomized humidification liquid in the humidified gas after the humidified gas enters a delivery conduit and prior to the humidified gas being provided adjacent to or into the patient by the delivery conduit.

22. The method of claim 20, wherein providing the humidified gas adjacent to or into the patient comprises providing the humidified gas at a temperature range of 20-45° Celsius adjacent to or into the patient.

23. The method of claim 20, further comprising:
providing the humidification liquid into a humidification chamber prior to atomizing the at least a portion of the humidification liquid;
providing a medicant into the humidification chamber, wherein the medicant is combined into the humidification liquid prior to atomizing the at least a portion of the humidification liquid; and
wherein an amount of the humidification liquid provided into the humidification chamber is controlled independently from an amount of the medicant provided into the humidification chamber.

24. The method of claim 23, wherein the amount of the medicant provided into the humidification chamber is independently controlled based on a medicant profile.

25. The method of claim 20, wherein the humidification liquid further comprises one or more medicants; and
wherein the method further comprises, after the humidified gas enters the patient, filtering at least a portion of the one or medicants out of patient.
